(19) United States
(12) Patent Application Publication
(10) Pub. No.: US 2004/0019369 A1
(43) Pub. Date: Jan. 29, 2004

(54) Wireless functional electrical stimulation system

(76) Inventors: Michael Duncan, New South Wales (AU); Ian Bruinsma, New South Wales (AU); Zoran Milijasevic, New South Wales (AU); Andrew Barriskill, New South Wales (AU)

Correspondence Address: Finnegan, Henderson, Farabow,, Garrett & Dunner, L.L.P., 1300 I Street, N.W., Washington, DC, 20005-3315, US

(21) Appl. No.: 10/383,551

(22) Filed: Mar. 10, 2003

(30) Foreign Application Priority Data
Mar. 11, 2002 (AU) PS1015

(51) Int. Cl. A61N 1/18

(52) U.S. Cl. 607/46

(57) ABSTRACT

A wireless, multi-purpose functional electrical stimulation (FES) system 10 includes a plurality of implantable stimulator units 12. Each unit 12 is implanted, in use, at a particular site in a patient's body 14 for stimulating and/or monitoring that site. Each stimulator unit 12 includes a power source and a programmable microcontroller for controlling stimulation at its associated site. Each stimulator unit 12 further has a plurality of implantable transducer elements 22 connected to and in communication with the microcontroller. At least certain of the transducer elements 22operate as stimulating electrodes. A controller 16 is arranged, in use, externally of the patient's body 14 for supplying programming and control signals transcutaneously to each of the stimulator units 12 independently to effect stimulation of the site associated with the stimulator unit 12 being addressed at that time by the controller 16.

### FIELD OF THE INVENTION

This invention relates to a multi-purpose, functional electrical stimulation (FES) system. More particularly, the invention relates to a wireless, multipurpose FES system and to an implantable stimulator for use in such a system.

### BACKGROUND TO THE INVENTION

Neurological impairment, such as spinal cord injury (SCI), cerebral palsy (CP), urinary incontinence (UI) etc, can occur in people of any age, and can be due to any number of causes. SCI, in particular, is often caused by injuries sustained in accidents associated with motor vehicles, firearms, sports injuries, or the like. Many of the individuals who sustain such injuries are young male adults between the ages of 16 and 30 who, up to the point of the accident, have lead active and healthy lives.

In the USA, the prevalence of neurological impairment resulting from SCI is currently estimated at between 712 and 906 per million with the incidence of SCI being calculated at between about 30 and 40 per million. It is widely recognised that SCI has a large impact on society in general and is a sudden and irreversible change to an individual's quality of life.

In order to define SCI, it should be understood that an SCI is a traumatic lesion to the spinal cord and the associated nerves. Thirty-one spinal nerves originate from the spinal cord and can be grouped as follows: 8 cervical (C1 to C8), 12 thoracic (T1 to T12), 5 lumbar (L1 to L5), 5 sacral (S1 to S5) and 1 coccygeal. An injury to the spinal cord can result in varying degrees of impairment depending on where and to what extent the spinal cord is injured. In general, the higher up on the spinal cord the injury, the more severe the resulting impairment.

People suffering from an SCI are essentially categorised into two main groups: tetraplegics and paraplegics.

Tetraplegics are individuals who have sustained an injury to one of the eight cervical segments of the spinal cord, C1 to C8. Such an injury results in impaired use of the arms and hands as well as the legs. A person who has suffered such an injury generally experiences significant loss of sensation and volitional body movement as well as the loss of volitional bladder and bowel control. Many tetraplegics may also have loss of psychogenic and impaired reflex erections.

Paraplegics are individuals who have sustained an injury at the thoracic level, T1 to T12. These individuals usually have sensation and volitional control over their upper limbs, but have lost sensation and control of their lower limbs and bladder and bowel control, as well as erection problems in males.

Due to SCI individuals being unable to control bladder function, individuals must regularly self cathertise. This procedure is problematic, especially for females, and can result in an increase in the incidence of urinary tract infections. Still further, persons suffering from SCI must often undertake lengthy bowel evacuation procedures using, for instance, digital evacuation. SCI patients are also prone to secondary medical problems, such as pressure sores, osteoporosis, muscular atrophy in the lower limbs, muscle spasticity, deep vein thrombosis, cardiovascular disease and depression. Pressure sores are caused by the occlusion of blood flow during sitting and lying. They are a major health problem which may require surgery to repair and months of rehabilitation including requiring the patient to remain lying on their abdomen for an extended period of time.

Therefore, whilst restoration of bladder and bowel control is a primary need of SCI individuals, reduced incidence of pressure sores is also highly needed. This, together with the ability to exercise and stand and step, are functions that would greatly improve the quality of life of SCI individuals.

It is therefore evident that a large proportion of the population who have an SCI would benefit from a device that would be able to assist in the at least partial restoration of such lost functionality, in particular bowel and bladder function, erectile function, the reduction in the incidence of pressure sores and the provision of exercise and upright mobility. Various systems have been proposed by numerous organisations to deal with one or other of the functions that have been lost to SCI individuals.

In the applicant's co-pending International Patent Application No. PCT/AU03/00044, a multi-purpose, functional electrical stimulation system is disclosed. That patent application is specifically incorporated herein by reference. The system is used to stimulate a number of sites in a patient's body using a single stimulator unit.

More particularly, the stimulator unit is, in use, implanted in a costal region of the patient's body and may be required to stimulate regions such as the upper or lower extremities of the patient's body and the sacral and/or thoracic regions of the patient's spinal cord. Each site has multiple stimulation points which necessitates the leading of numerous electrical leads from the location of the stimulator unit to the relevant site.

As a development of the above invention, the applicant has subsequently filed International Patent Application No. PCT/AU03/00139 related to a distributed, multipurpose FES system and to a switching node for use in such a system. Once again, the teachings of International Patent Application No. PCT/AU03/00139 are incorporated herein by reference. The distributed system reduces the number of leads which are required to be implanted in a patient's body thereby reducing the risk of the spread of infection, the invasive nature of the implanting procedure and discomfort to the patient.

Various wireless FES systems using single channel injectable microstimulators have been proposed. (These systems are referred to below as “microstimulator systems”.) Such microstimulator systems are disclosed, for example, in U.S. Pat. No. 5,324,316 to Schulman et al, U.S. Published Patent Application No. 2001/0037132 to Whitehurst et al and US Published Patent Application No. 2001/0001125 to Schulman et al. All of these systems suffer from the drawback that a number of individually addressable stimulators have to be injected into each site. A control signal to effect a stimulation at the relevant site therefore has to be a complicated signal containing not only addressing data for all the microstimulators at the site but also the stimulation data for each microstimulator and a power component for each stimulator. Also, because the microstimulators are not secured to tissue, there is the possibility that they can dislodge or migrate from their required positions resulting in inaccurate operation of the microcontroller systems with the resultant risks.

The applicant now proposes a system which further reduces the number of electrical leads required to be implanted in a patient's body.

### SUMMARY OF THE INVENTION

According to a first aspect of the invention, there is provided a wireless, multipurpose functional electrical stimulation (FES) system which includes

a plurality of implantable stimulator units, each unit being implanted, in use, at a particular site in a patient's body for at least one of stimulating and monitoring that site, each stimulator unit including a power source and a programmable control means for controlling stimulation at its associated site, each stimulator unit further having a plurality of implantable transducer elements connected to and in communication with the programmable control means, at least certain of the transducer elements operating as stimulating electrodes; and

a controller arranged, in use, externally of the patient's body for supplying programming and control signals transcutaneously to each of the stimulator units independently to effect stimulation of the site associated with the stimulator unit being addressed at that time by the controller.

The system may stimulate any number of sites in the patient's body. These sites may include a right upper extremity or lower extremity of the patient's body, a left upper extremity or lower extremity of the patient's body and a sacral/posterior region of a patient's spinal cord. Those skilled in the art will, however, appreciate that the number of sites to be stimulated will be dependent entirely on the type of disability for which the system seeks to compensate and/or, in the case of spinal cord injury (SCI), the level of severity of the SCI.

The number of stimulating electrodes connected to each stimulating unit may be governed by the number of stimulation points required to cause effective stimulation at the site. Typically, each stimulator unit may have connectors for allowing connection of up to six electrodes, the stimulating electrodes being in communication with the control means of their associated stimulating unit via a switching arrangement.

Preferably, certain other transducer elements of each stimulator unit function as measurement sensors so that the stimulator unit is also used for making biomedical measurements and measurements of physical parameters at its associated site. Therefore, by appropriate interrogation of the stimulator unit by the controller, each stimulator unit can be used for effecting biomedical sensing functions at the site, the biomedical information being sent by the stimulator unit to the controller.

The stimulator units may be addressed by the controller individually by means of an appropriate addressing technique, such as time division multiplexing. Instead, each stimulator unit may have a unique address associated with it which is used by the controller for addressing that specific stimulator unit. Still further, each stimulator unit may be addressed using a different frequency in the relevant stimulation frequency range.

The power source of each stimulator unit may include a battery. Preferably, the battery is a rechargeable battery.

The system may include an indicating means for indicating to the controller which stimulator unit requires charging and a charging device for charging the battery of each stimulator unit, for example, when a patient is undergoing a sleep cycle or when the patient is in an appropriate electrical field. Conveniently, the charging device is incorporated in the controller of the system. It will, however, be appreciated that the charging device could be a separate unit or could be implemented as a localised charging device, such as a charging coil, placed in close proximity to the stimulator units, for example, by being mounted on a patient's bed or wheelchair.

Each stimulator unit may include a power receiving device for receiving a recharging signal from the charging device. The power receiving device may be in the form of a charging antenna which is configured to receive electromagnetic charging power from the charging device. The charging antenna may recharge the battery of the power source via a charging circuit in the stimulator unit, the charging circuit operating under control of the control means of the stimulator unit.

Each stimulator unit may receive stimulator instructions from the controller via a wireless data link. The data link may be a radio frequency (RF) link.

Further, each stimulator unit may include a transmitter/receiver (transceiver) device for receiving data signals from the controller and, where applicable, transmitting data signals to the controller. The transceiver device may be in the form of an RF antenna which communicates with the control means of the stimulator unit via an RF transceiver unit. The transceiver device and the charging antenna may be implemented as a single device.

The control means of each stimulator unit may include a memory means which receives and stores a control algorithm containing data relevant to a stimulation regime from the controller. This enables the system to operate in an autonomous mode, or autonomously in co-ordination with other stimulators. For example, one of the stimulator units may be a master unit with the remaining stimulator units being slave units. The controller may address the master unit and the master unit, in turn and where applicable, may then address the relevant slave stimulator unit at the site to be stimulated.

According to a second aspect of the invention, there is provided an implantable stimulator unit for use in a wireless, multi-purpose functional electrical stimulation (FES) system, the stimulator unit including

a power source;

a control means which receives power from the power source and which receives data signals from an external controller of the system; and

a plurality of implantable transducer elements connected to and in communication with the programmable control means, at least certain of the transducer elements operating as stimulating electrodes.

The number of transducer elements functioning as stimulating electrodes may be governed by the number of stimulation points required to cause effective stimulation at the site. The transducer elements may be in communication with the control means via a switching arrangement.

The stimulator unit may function as a measurement unit for making biomedical measurements and measurements of physical parameters at its associated site, at least certain other transducer elements being measurement sensors to provide measurement data to the control means.

The stimulator unit may also function as a self diagnostic unit capable of determining the status of its various components and for transmitting such status data to the controller. Such a feature may prove useful in providing data such as charge state of the power source of the stimulator unit to the controller, thereby ensuring that the unit is in a constant state of operational readiness.

The power source may include a battery. The battery may be a rechargeable battery. The stimulator unit may include a power receiving device for receiving a recharging signal from a charging device of the system. The power receiving device may be in the form of a charging antenna which is configured to receive electromagnetic charging power from the charging device. The charging antenna may recharge the battery of the power source via a charging circuit, the charging circuit operating under control of the control means.

The unit may receive data from the controller via a wireless data link.

The unit may include a transmitter/receiver (transceiver) device for receiving data signals from the controller and, where applicable, transmitting data signals to the controller.

The control means may include a memory means which receives and stores a control algorithm containing data relevant to a stimulation regime.

### BRIEF DESCRIPTION OF THE DRAWINGS

The invention is now described by way of example with reference to the accompanying diagrammatic drawings in which:

FIG. 1 shows a block diagram of a wireless, multi-purpose functional electrical stimulation system, in accordance with a first aspect of the invention;

FIG. 2 shows a block diagram of a stimulator unit, in accordance with a second aspect of the invention, for use in the system of FIG. 1; and

FIG. 3 shows a simplified view of one embodiment of the system of FIG. 1 following surgical implantation.

### DETAILED DESCRIPTION OF THE DRAWINGS

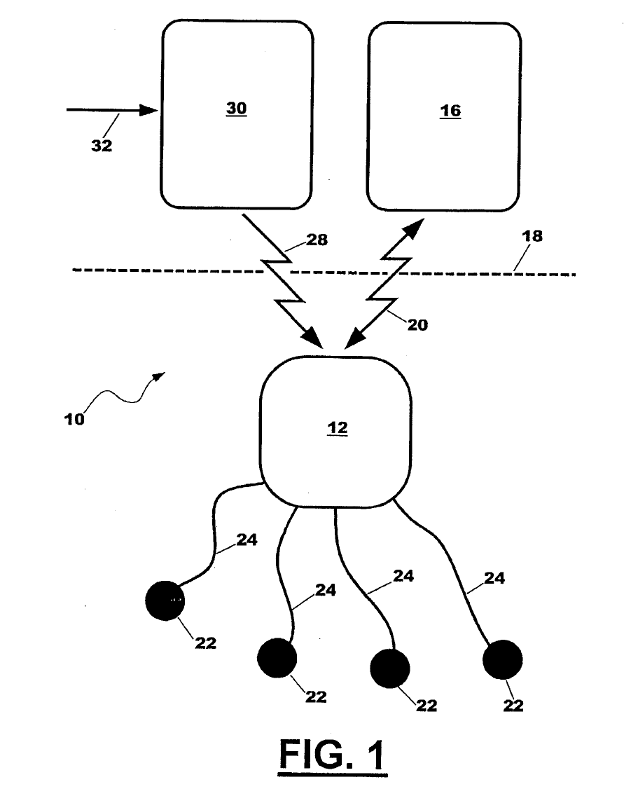

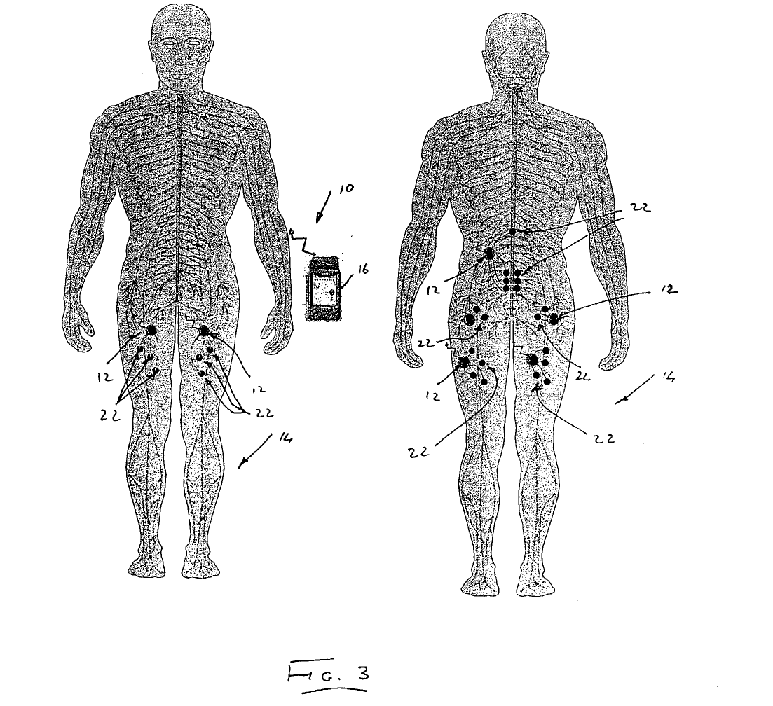

In the drawings, reference numeral 10 generally designates a wireless, multipurpose functional electrical stimulation (FES) system, in accordance with a first aspect of the invention. The system 10 includes a plurality of stimulator units 12, one of which is shown in FIG. 1 of the drawings. As shown in FIG. 3 of the drawings, a preferred implementation of the system 10 includes multiple stimulator units 12 implanted in a patient's body 14.

The system 10 includes a single controller 16 which is arranged externally of the patient's body 14. The controller 16 communicates with each of the stimulator units 12 transcutaneously as shown schematically in FIG. 1 of the drawings, where the skin of the patient's body 14 is represented at 18. The controller 16 communicates bi-directionally with each stimulator unit 12, as will be discussed in greater detail below and as represented by control signal 20. The controller 16 also communicates with other devices arranged externally of the patient's body 14. These externally arranged devices include external sensors, programming devices, communications devices, or the like.

Each stimulator unit 12 has a plurality of transducer elements 22 which are either stimulating electrodes or measurement sensors, as the case may be. Each element 22 is connected to the stimulator unit 12 by means of an electrical lead 24. In certain circumstances, each stimulator unit 12 also conducts biomedical measurements and sensing and feeds this data via the bi-directional control signal 20 to the controller 16. To enable these measurements and/or sensing applications to occur, at least certain of the transducer elements 22 are therefore dedicated measurement sensors.

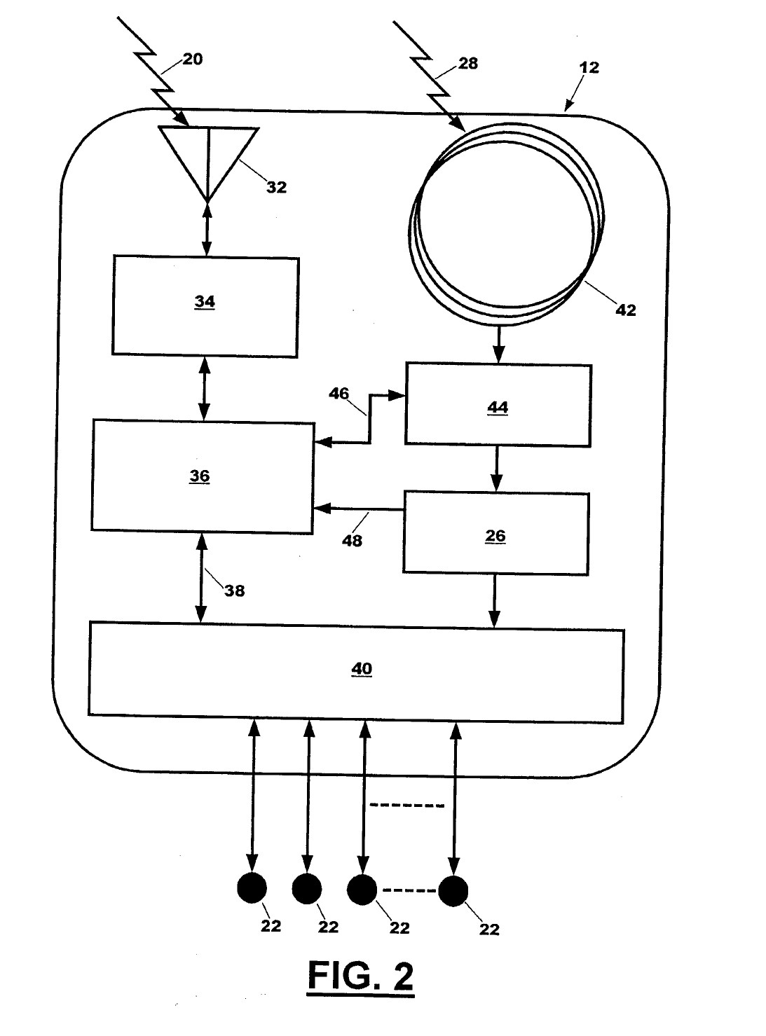

Because the system 10 functions as a distributed system with a plurality of discrete stimulator units 12, each stimulator unit 12 includes its own power source 26 (FIG. 2). The power source 26, conveniently, includes a rechargeable battery. The power source 26 receives a charging signal 28 from a charger 30 arranged externally of the patient's body 14. The charger 30, in turn, receives power from an external power supply as represented by arrow 32 in FIG. 1 of the drawings.

The charger 30 is in the form of an electric field generating means which, when the patient passes through it, or is in that field, causes the battery of the power source 26 of each stimulator unit 12 to be recharged. The charger 30 may also include the use of localised power coils that are placed in close proximity to individual stimulator units 12 to facilitate localised, more intense charging conditions of such stimulator units 12. Further, the charger 30 could be implemented as a signal generating device which generates the signal 28 to charge the stimulator units 12, for example, when the patient is at rest such as when undergoing a sleep cycle or when seated in a wheelchair.

In the latter case, the charger 30 could be incorporated into the controller 16 to form a single unit.

Referring now to FIG. 2 of the drawings, a stimulator unit 12, in accordance with a second aspect of the invention, is described in greater detail. Each stimulator unit 12 incorporates a receiving device in the form of an RF antenna 32. The RF antenna 32 feeds the received control signal 20 to an RF transceiver unit 34. The RF transceiver unit 34 communicates with a programmable control means in the form of a programmable microcontroller or logic 36. The microcontroller 36, in turn, feeds control signals 38 to a switching unit 40. The switching unit 40 is a programmable unit which interfaces the transducer elements 22 with the microcontroller 36. The switching unit 40 controls electrode stimulation current level, electrode selection and supplies one or more connections from a transducer element 22 operating as a measurement sensor to the microcontroller 36.

Each stimulator unit 12 further includes a power receiving device in the form of a charging antenna 42. The charging antenna 42 receives the charging signal 28 from the charger 30 and feeds it to the battery of the power source 26 of the stimulator unit 12 via a charging circuit 44. The charging signal 28 can either be a high frequency electromagnetic signal or a low frequency magnetic signal.

Further, the charging antenna 42 could be implemented with the receiving antenna 32 as a single device.

The charging circuit 44 converts the charging signal 28 received by the charging antenna 42 into a form suitable to charge the battery of the power source 26. The charging circuit 44 also supplies charging status to the microcontroller 36. The microcontroller 36 communicates, in a bi-directional manner, with the charging circuit 44 as illustrated by signal line 46. It is also to be noted that a status of the power source 26 is fed to the microcontroller 36 via an indicating means in the form of a signal line 48. The status of the power source 26 is, therefore, able to be transmitted from the stimulator unit 12 to the controller 16 so that the controller 16 is able to charge only those stimulator units 12 which transmit to the controller 16 a request for a charging signal 28.

The microcontroller 36 is a programmable unit and is programmed remotely using the controller 16 via the data link 20. The controller 36 transmits and receives digital status and control data via the RF link 20. It also, as described above, controls the charging circuit 44 and monitors the battery status of the power sources 26.

Although not shown, the microcontroller 36 includes a separate memory means wherein stimulation patterns and the like are stored to be accessed by the microcontroller 36. In such instances a stimulation routine is downloaded from the controller 16 and stored in the memory means. In response to a measured condition or time trigger, the stimulator unit 12 implements the stored stimulation routine to perform a desired task without the need to receive specific instructions from the controller 16.

As described above, when certain of the transducer elements 22 are measurement sensors, data from these sensors are fed via the switching unit 40 to the microcontroller 36. The microcontroller 36 feeds the data received from the measurement sensors via the transceiver unit 34 and RF antenna 32 to the controller 16. If desired, the data are stored in the memory of the microcontroller 36. Such data can then be accessed by the microcontroller 36 so that stimulation instructions are fed to the stimulating electrodes without the need for reception of data from the controller 16. Therefore the stimulating unit 12 can operate in an autonomous mode in certain circumstances.

The stimulator unit 12 also functions as a self diagnostic unit capable of determining the status of its various components and for transmitting such status data to the controller 16. Thus, data such as a charge state of the power source 26 of the stimulator unit 12 can be transmitted by the microcontroller 36 of the stimulator unit 12 to the controller 16, thereby ensuring that the unit is in a constant state of operational readiness. In addition, the status of other components can also be transmitted to the controller 16 so that, if there is a malfunction in any such component, remedial action can be taken by a clinician.

In use, each site in the patient's body 14 to be stimulated has a stimulator unit 12 implanted therein or in close proximity thereto. Transducer elements 22 are connected to the stimulator unit 12 and the transducer elements 22 are sutured, or otherwise secured, at predetermined stimulation/measurement points to tissue, be it nerve tissue or muscle tissue, at the site. While FIG. 3 illustrates the stimulator units 12 being associated with the spinal cord, the right lower extremity and left lower extremity of the patient's body 14, it is envisaged that stimulator units 12, with their associated transducer elements 22 could also be used for any stimulation/measurement purposes in the patient's body 14 including the upper extremities, deep brain stimulations such as for Parkinson's disease sufferers, cerebral palsy patients, sleep apnoea, dorsi-flexure in stroke patients, or the like.

The system 10 operates in a similar manner to the applicant's previously described systems in that, being a multi-purpose FES system, the system 10 stimulates multiple sites in the patient's body 14. Thus, for example in the case of a paraplegic/tetraplegic person that person can use the system 10 to aid in exercising a measure of control over the particular sites, more particularly, the right lower extremity, the left lower extremity and the sacral and/or thoracic regions of the spinal cord of the patient's body 14. By using the system 10 the patient can implement standing/stepping and sitting strategies. In respect of the spinal cord regions which are stimulated, bladder control, bowel control and erectile dysfunction (in the case of male patients) strategies can be effected. The system 10 can also be used for effecting strategies in regard to the upper extremities of the patient's body 14 and for effecting appropriate strategies in patients with brain diseases or brain injuries. Still further, the system 10 can be used for treating shoulder subluxation and for pain management.

Due to the programmable nature of the stimulator units 12, one or more stimulator units 12 may be programmed to generate stimulation sequences independently, whether continuously, in a time based manner, or in response to information from a connected measurement sensor 22. Still further one of the stimulator units 12 can be designated as a master unit with the remaining stimulator units 12 being slave units. The master unit is the stimulator unit 12 addressed by the controller 16. The master unit, if it is not the site associated with the master unit that is to be stimulated, then transmits the stimulation data wirelessly to the relevant slave stimulator unit at the site to be stimulated. This therefore further reduces the complexity of the control signal 20 from the controller 16.

It will be appreciated that each system needs to be tailored individually for the patient. This is readily accommodated by the wireless system 10 of the present invention. In addition, the system 10 is scalable and easily adaptable in the sense that the stimulator units 12 of the system 10 can be implanted in stages. Thus, the surgical procedure can be broken down into more manageable stages where, for example, first the bladder/neuromodulation group of stimulator units 12 are implanted in one procedure followed, at a later stage, by the implantation of the stimulator units 12 associated with the right lower extremity and the left lower extremity of the patient's body 14.

Still further, it is an advantage of the invention that the system 10 is less invasive due to the absence of leads from a central stimulator unit to the sites of the patient's body. A related advantage of this aspect of the invention is that no leads need to be subcutaneously tunnelled from a centralised stimulator to the sites of the patient's body 14. This obviates the need for extra incisions which are used to facilitate tunnelling.

Also, because there are fewer leads in the patient's body and all elements of the implanted system do not require physical connection to each other, the patient's body 14 is less prone to infection. It will be appreciated that in a system in which all elements are physically connected together, an infection at one part of the system can quickly travel along connecting leads, where such leads are used, necessitating removal of the entire system. The system 10 obviates this problem to a large extent.

Still further, if there is a failure of any one stimulator unit 12 this can be dealt with locally at that site of the patient's body 14 and it is not necessary to replace the whole system 10.

A further advantage of the present invention is that it provides considerably more freedom to the user, in comparison with other systems of which the applicant is aware. As there is no longer a requirement for the stimulator units to remain in direct communication with the controller to receive power and data, traditionally via a proximal antenna, the programmable nature of the stimulator units 12 allows the system 10 to be used in environments not previously thought possible. Such environments may include showering, washing or swimming by the patient, where previously an external electronic controller was unable to operate reliably. This additional flexibility is also advantageous in terms of using the system 10 to assist in providing erectile function. In such instances the user would be able to operate the system 10 without the need for external controller wires, resulting in a more natural experience.

Still further, the present invention provides a more reliable system than more traditional systems. Such traditional systems rely upon the power and data being transmitted to the implanted stimulator via a strategically positioned antenna. In the event of the external transmitting antenna being incorrectly aligned or temporarily dislodged to impair direct communication with the receiving antenna of the implanted stimulator, the system could operate in an unsafe/unreliable manner. In the present invention, as there is no need for a direct antenna-antenna alignment for data and/or power transfer, the reliability of the system is improved.

It is yet a further advantage of the system 10 that only a single packet of information needs be transmitted to the relevant stimulator unit 12 to carry out a stimulation strategy at that site. This is unlike the case with systems employing a plurality of microstimulators at each site where the microstimulators have to be addressed. powered and controlled individually. Thus, the complexity of the control signal in the case of the present invention is substantially reduced in comparison with such microcontroller systems. Therefore, the likelihood of spurious or incorrect of operation of the system of the present invention is much lower than in the case of microstimulator systems. Also, because the transducer elements 22 of the present invention are sutured to the tissue, there is less likelihood of the elements 22 being displaced than in the case of the microstimulators of the microstimulator systems. Once again, this reduces the risks associated with incorrect stimulation of a site.

In addition, the system 10 can, in effect, operate autonomously in the sense that the relevant stimulating unit 12 can respond to data from its associated measurement sensors. This further improves the versatility of the system 10.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

We claim:

1. A wireless, multi-purpose functional electrical stimulation (FES) system which includes
a plurality of implantable stimulator units, each unit being implanted, in use, at a particular site in a patient's body for at least one of stimulating and monitoring that site, each stimulator unit including a power source and a programmable control means for controlling stimulation at its associated site, each stimulator unit further having a plurality of implantable transducer elements connected to and in communication with the programmable control means, at least certain of the transducer elements operating as stimulating electrodes; and
a controller arranged, in use, externally of the patient's body for supplying programming and control signals transcutaneously to each of the stimulator units independently to effect stimulation of the site associated with the stimulator unit being addressed at that time by the controller.

2. The system of claim 1 in which the number of stimulating electrodes connected to each stimulating unit is governed by the number of stimulation points required to cause effective stimulation at the site.

3. The system of claim 2 in which the stimulating electrodes are in communication with the control means of their associated stimulating unit via a switching arrangement.

4. The system of claim 1 in which certain other transducer elements of each stimulator unit function as measurement sensors so that the stimulator unit is also used for making biomedical measurements and measurements of physical parameters at its associated site.

5. The system of claim 1 in which the stimulator units are addressed by the controller individually by means of an addressing technique.

6. The system of claim 1 in which the power source of each stimulator unit includes a battery.

7. The system of claim 6 in which the battery is a rechargeable battery.

8. The system of claim 7 which includes an indicating means for indicating to the controller which stimulator unit requires charging and a charging device for charging the battery of each stimulator unit.

9. The system of claim 8 in which the charging device is incorporated in the controller of the system.

10. The system of claim 8 in which each stimulator unit includes a power receiving device for receiving a recharging signal from the charging device.

11. The system of claim 10 in which the power receiving device is in the form of a charging antenna which is configured to receive electromagnetic charging power from the charging device.

12. The system of claim 11 in which the charging antenna recharges the battery of the power source via a charging circuit in the stimulator unit, the charging circuit operating under control of the control means of the stimulator unit.

13. The system of claim 1 in which each stimulator unit receives stimulator instructions from the controller via a wireless data link.

14. The system of claim 13 in which each stimulator unit includes a transmitter/receiver (transceiver) device for one of receiving data signals from the controller and transmitting data signals to the controller.

15. The system of claim 1 in which the control means of each stimulator unit includes a memory means which receives and stores a control algorithm containing data relevant to a stimulation regime from the controller.

16. The system of claim 1 in which one of the stimulator units is a master unit and the remaining stimulator units are slave units.

17. The system of claim 16 in which the controller addresses the master unit and the master unit, in turn and where applicable, addresses the relevant slave stimulator unit at the site to be stimulated.

18. An implantable stimulator unit for use in a wireless, multi-purpose functional electrical stimulation (FES) system, the stimulator unit including a power source;
a control means which receives power from the power source and which receives data signals from an external controller of the system; and
a plurality of implantable transducer elements connected to and in communication with the programmable control means, at least certain of the transducer elements operating as stimulating electrodes.

19. The unit of claim 18 in which the number of transducer elements functioning as stimulating electrodes is governed by the number of stimulation points required to cause effective stimulation at the site.

20. The unit of claim 18 in which the transducer elements are in communication with the control means via a switching arrangement.

21. The unit of claim 18 which functions as a measurement unit for making biomedical measurements and measurements of physical parameters at its associated site, at least certain other transducer elements being measurement sensors to provide measurement data to the control means.

22. The unit of claim 18 which functions as a self diagnostic unit capable of determining the status of its various components and for transmitting such status data to the controller.

23. The unit of claim 18 in which the power source includes a battery.

24. The unit of claim 23 in which the battery is a rechargeable battery.

25. The unit of claim 24 which includes a power receiving device for receiving a recharging signal from a charging device of the system.

26. The unit of claim 25 in which the power receiving device is in the form of a charging antenna which is configured to receive electromagnetic charging power from the charging device.

27. The unit of claim 26 in which the charging antenna recharges the battery of the power source via a charging circuit, the charging circuit operating under control of the control means.

28. The unit of claim 18 which receives data from the controller via a wireless data link.

29. The unit of claim 28 which includes a transmitter/receiver (transceiver) device for one of receiving data signals from the controller and transmitting data signals to the controller.

30. The unit of claim 18 in which the control means includes a memory means which receives and stores a control algorithm containing data relevant to a stimulation regime.
